SEQUENCE LISTING (5 sequences)

SEQ ID NO: 1 - DNA, 15 bp, synthetic construct
  source 1..15: mol_type = other DNA
cagacgtgtgctctt

SEQ ID NO: 2 - DNA, 27 bp, synthetic construct
  source 1..27: mol_type = other DNA
nnnnnnnagatcggaagagcgtcgtgt

SEQ ID NO: 3 - RNA, 44 nt, synthetic construct
  source 1..44: mol_type = other RNA
rargrarnrcrgrgrarargrargrcrarcrarcrgrnrcrnrg

SEQ ID NO: 4 - DNA, 21 bp, synthetic construct
  source 1..21: mol_type = other DNA
agacgtgtgctcttccgatct

SEQ ID NO: 5 - DNA, 21 bp, synthetic construct
  source 1..21: mol_type = other DNA
tacacgacgctcttccgatct

(19) United States
(12) Patent Application Publication
(10) Pub. No.: US 2026/0250666 A1
(43) Pub. Date: Aug. 27, 2026

(54) METHODS FOR OBTAINING RNA SECONDARY STRUCTURE OF LNP-ENCAPSULATED RNA

(71) Applicant: ECLIPSE BIOINNOVATIONS, INC., San Diego, CA (US)

(72) Inventors: Anide Johansen, San Diego, CA (US); Stephanie C. Bruns, San Diego, CA (US); Dayanara Lebron-Aldea, San Diego, CA (US); Karen B. Chapman, San Diego, CA (US)

(21) Appl. No.: 18/870,946

(22) PCT Filed: Jun. 1, 2023
(86) PCT No.: PCT/US2023/067794

§ 371 Date: Dec. 2, 2024

Related U.S. Application Data
(60) Provisional application No. 63/348,738, filed on Jun. 3, 2022.

(51) Int. Cl. C12N 15/10

(52) U.S. Cl. CPC C12N 15/1093; C12N 15/1096

(57) ABSTRACT

The present disclosure relates to methods of obtaining RNA secondary structure by assessing nucleotide flexibility of an RNA molecule within a lipid nanoparticle (LNP). Some embodiments include contacting an LNP-encapsulated RNA sample with one or more structure probing agents, isolating at least one RNA from the LNP, and sequencing the isolated RNA or cNDA generated from the RNA.

### CROSS-REFERENCE TO RELATED APPLICATIONS

This patent application is a U.S. National Phase of International Application No. PCT/US2023/067794, filed Jun. 1, 2023, which claims the benefit of U.S. Provisional Application No. 63/348,738 filed on Jun. 3, 2022, which is incorporated by reference in its entirety.

### BACKGROUND

The specific secondary structures formed by non-coding and messenger RNAs are central to their regulation, influencing gene transcription, translation, and decay. RNA has emerged as a new category of therapeutic agents to prevent and treat various diseases. To function in vivo, RNA molecules require safe, efficient, and stable delivery to protect the molecule from degradation and to aid intracellular delivery. One such delivery system is the lipid nanoparticle (LNP), a spherical vesicle made of ionizable lipids. Selective 2′-Hydroxyl Acylation analyzed by Primer Extension (SHAPE) is a chemical probing method that measures RNA flexibility at single nucleotide resolution. Nucleotide flexibility can further be used to obtain RNA secondary structure. SHAPE technology has so far been applied to RNA molecules in cells (in vivo) and in solution (in vitro) but has yet to be applied to LNP-encapsulated RNA molecules.

### REFERENCE TO SEQUENCE LISTING

The present application is filed with a Sequence Listing in Electronic format. The Sequence Listing is provided as a file entitled EBIO.009WO_ST 26.xml, created May 31, 2023, which is approximately 6 kb in size. The information in the electronic format of the sequence listing is incorporated herein by reference in its entirety.

### SUMMARY

In aspects, the disclosure relates to a method of obtaining RNA secondary structure by assessing nucleotide flexibility of a lipid nanoparticle (LNP) encapsulated RNA molecule. In some embodiments, the method comprises contacting an LNP-encapsulated RNA sample with a structure probing agent, isolating the RNA from the LNP, ligating the RNA molecule to an adapter, amplifying RNA molecules, or cDNA molecules thereof, by PCR, sequencing the PCR products, and identifying computationally RNA molecules. In some embodiments, the method may be used to determine RNA secondary structure while encapsulated in an LNP. In some embodiments, the structure probing agent may modify the RNA sugar backbone, all four bases, or be base selective. In some embodiments, the structure probing agent may be NAI or 2A3.

Another embodiment may include a method of obtaining RNA secondary structure by assessing nucleotide flexibility of an RNA molecule within a lipid nanoparticle (LNP), the method comprising: a) contacting an LNP-encapsulated RNA sample with one or more structure probing agents; b) isolating at least one RNA or cDNA from the LNP-encapsulated RNA sample; and c) sequencing the isolated RNA or cDNA generated from the at least one RNA or cDNA; and d) obtaining RNA secondary structure by assessing nucleotide flexibility of the LNP-encapsulated RNA sample.

Another embodiment is a kit that includes one or more structure probing agents; and a manual providing instructions for obtaining RNA secondary structure of an LNP-encapsulated RNA sample.

These and other features, aspects, and advantages of the present disclosures will become better understood with reference to the following description, drawings, and claims.

### BRIEF DESCRIPTION OF DRAWINGS

FIG. 1 illustrates a schematic diagram depicting an embodiment of a protocol for obtaining secondary structure of an LNP-encapsulated RNA molecule.

FIG. 2A illustrates mutation rates from NAI/DMSO treated samples, where the RNA is derived from LNP-encapsulated RNA and free RNA. FIG. 2B illustrates coverage, mutation rates and SHAPE reactivity plots for NAI or DMSO treated LNP-RNA samples.

FIG. 3 illustrates RNA integrity as measured by Agilent TapeStation after RNase A treatment of LNP-RNA in an intact sample (LNP protects RNA from degradation) and compromised sample (Triton-X).

### DETAILED DESCRIPTION

In the Summary Section and Drawings Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition, kit, or device, the term “comprising” means that the compound, composition, kit, or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

All references cited herein, including but not limited to published and unpublished applications, patents, and literature references, are incorporated herein by reference in their entirety and are hereby made a part of this specification.

Where a range of values is provided, it is understood that the upper and lower limit, and each intervening value between the upper and lower limit of the range is encompassed within the embodiments.

### Methods

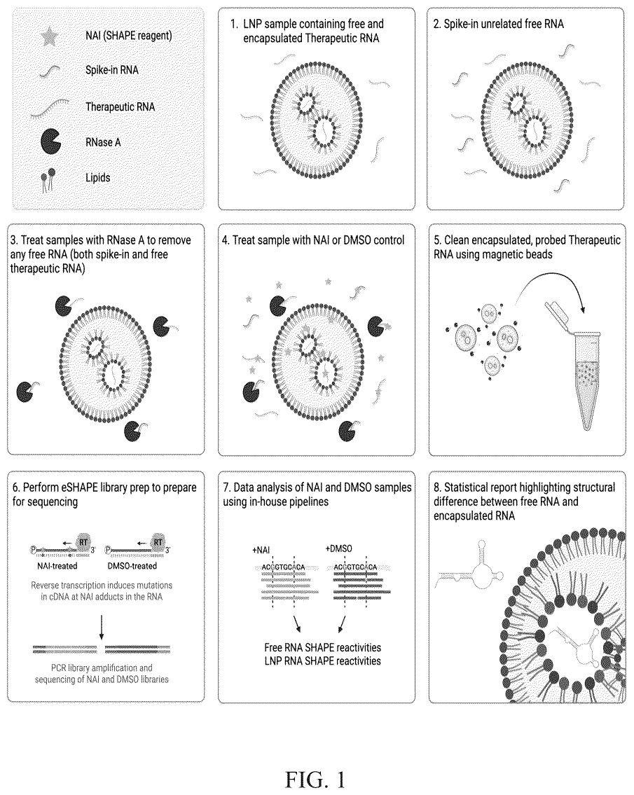

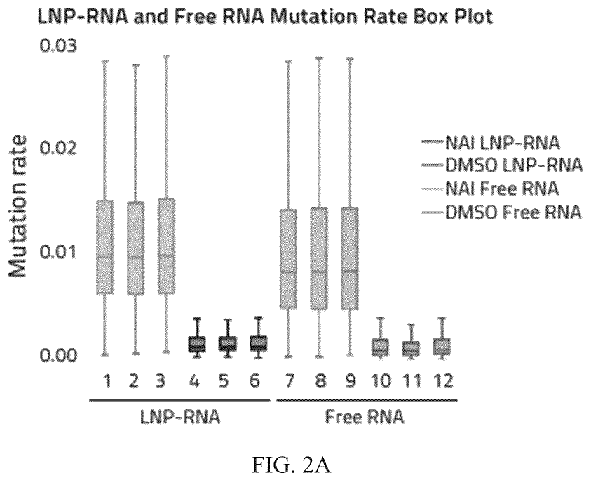

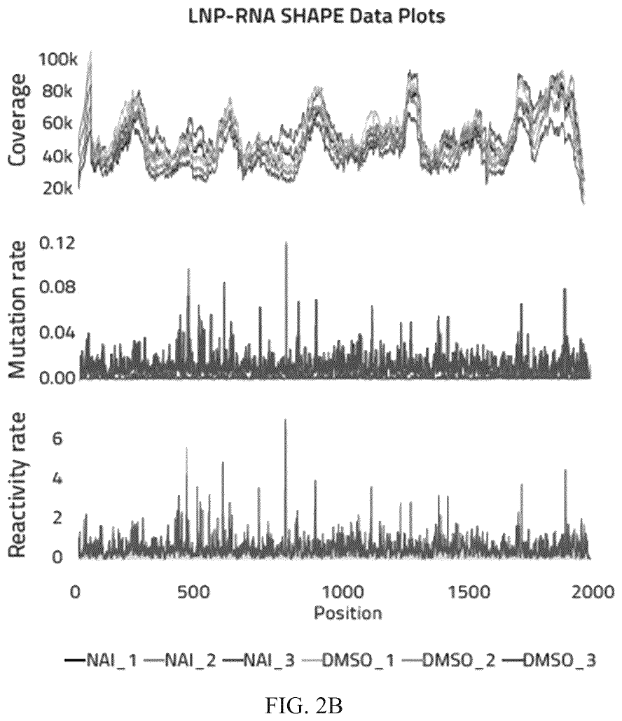

In aspects, the disclosure relates to a method of obtaining RNA secondary structure by assessing nucleotide flexibility of a lipid nanoparticle (LNP) encapsulated RNA molecule (FIG. 1). In some embodiments, the method includes contacting the LNP-encapsulated RNA sample with a structure probing agent. In some embodiments, the structure probing agent may be selected from, but not limited to, NAI, NAI-N3, DMS, 1M7, 1M6, NMIA, 5NIA, or 2A3. In some embodiments, the LNP-encapsulated RNA sample may include a polyA selected RNA, in vitro transcribed RNA, or a synthetic RNA sample. In some embodiments, the method further comprises isolating the RNA sample from the LNP. In some embodiments, the method further comprises forming cDNA through reverse transcription. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction (FIG. 2). In some embodiments, the method further comprises computing nucleotide flexibility.

In some embodiments, the method comprises contacting the LNP-encapsulated RNA sample with a structure probing agent. In some embodiments, the LNP-encapsulated RNA sample may comprise a linear or circular RNA. In some embodiments, the LNP-encapsulated RNA sample may comprise mRNA. In some embodiments, the LNP-encapsulated RNA sample is from mammal cells. In some embodiments, the LNP-encapsulated RNA sample is from bacterial cells. In some embodiments, the LNP-encapsulated RNA sample is from viral particles. In some embodiments, the LNP-encapsulated RNA sample is polyA-selected, ribosomal RNA depleted, in vitro transcribed or of synthetic origin. In some embodiments, the LNP-encapsulated RNA sample includes one or more modified nucleic acids. In some embodiments, the one or more modified nucleic acids is of synthetic origin. In some embodiments, the one or more modified nucleic acids of synthetic origin is selected from, but not limited to, locked nucleic acid (LNA), pseudouridine, 1-methyl-pseudouridine, 6-methyl-adensoine, 2′ methyl, 2′-O-(2-methoxyethyl). In some embodiments, the RNA sample includes a synthetic nucleic acid. In some embodiments, the synthetic nucleic acid is a locked nucleic acid (LNA). In some embodiments, the method further comprises isolating the RNA sample from the LNP. In some embodiments, the method further comprises linearizing a circular RNA, if the LNP-encapsulated RNA is circular. In some embodiments, the method further comprises formation of cDNA through reverse transcription. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction. In some embodiments, the method further comprises computing nucleotide flexibility.

In aspects, the disclosure relates to a method of obtaining RNA secondary structure for an LNP-encapsulated RNA sample. In some embodiments, the method comprises contacting the LNP-encapsulated RNA sample with a structure probing agent. The RNA sample may comprise a coding RNA or a non-coding RNA. In some embodiments, the method further comprises isolating the RNA sample from the LNP. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction. In some embodiments, the method further comprises computing nucleotide flexibility.

In some embodiments, the method further comprises formation of cDNA through reverse transcription. In some embodiments, the reverse transcription step precedes an enrichment step. In some embodiments, the reserve transcription step includes a buffer. In some embodiments, the reverse transcription buffer includes manganese. In some embodiments, the reverse transcriptase is selected from, but not limited to, SSII, SSIII, SSIV, TGIRT-III, MarathonRT, Maxima H, or combinations thereof.

In some embodiments, the method comprises contacting the LNP-encapsulated RNA sample with a structure probing agent. In some embodiments, the structure probing agent is capable of modifying the RNAs 2′ OH. In some embodiments, the structure probing agent is capable of modifying one or more of the RNAs nitrogenous bases. In some embodiments, the structure probing agent is selected from, but not limited to, NAI, NAI-N3, DMS, 1M7, 1M6, NMIA, 5NIA, 2A3, or combinations thereof. In some embodiments, the structure probing agent is quenched prior to isolation of the RNA from the LNP-encapsulated RNA sample. In some embodiments, the LNP-encapsulated RNA sample is treated with RNase. In some embodiments, a separate RNA is spiked into the LNP-encapsulated RNA sample before treating with RNase to monitor digestion of RNAs not LNP-encapsulated.

In some embodiments, the LNP-encapsulated RNA sample may comprise a modified or unmodified RNA. In some embodiments, the method further comprises isolating the RNA sample from the LNP. In some embodiments, the method further comprises linearizing a circular RNA, if the LNP-encapsulated RNA is circular. In some embodiments, the method further comprises formation of cDNA through reverse transcription. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction. In some embodiments, the method further comprises computing nucleotide flexibility.

In aspects, the disclosure relates to a method of obtaining RNA secondary structure for an LNP-encapsulated RNA sample. In some embodiments, the method comprises contacting the LNP-encapsulated RNA sample with a structure probing agent. The structure probing agent may modify the RNA sugar backbone or any of the RNA bases, or a combination thereof. In some embodiments, the method further comprises isolating the RNA sample from the LNP. In some embodiments, the method further comprises formation of cDNA through reverse transcription. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction. In some embodiments, the method further comprises computing nucleotide flexibility.

In some embodiments, the method comprises contacting the LNP-encapsulated RNA-RBP (RNA-binding protein) sample with a structure probing agent. In some embodiments, the method further comprises isolating the RNA sample from the LNP. In some embodiments, the method further comprises formation of cDNA through reverse transcription. In some embodiments, the method further comprises amplifying isolated RNA molecules, or cDNA molecules thereof. In some embodiments, the method further comprises sequencing the PCR products. In some embodiments, the method further comprises identifying computationally RNA molecules. In some embodiments, the method further comprises computing mutation rates and reactivities to guide RNA structure prediction. In some embodiments, the method further comprises computing nucleotide flexibility.

Some embodiments further include isolated RNA end repair. Some embodiments further include repairing RNA ends using FastAP, a phosphatase that removes 5′-phosphate from RNA-DNA chimeric molecules, and/or T4 PNK, which convert 2′-3′-cyclic phosphate to 3′-OH that is needed for further ligation. In some embodiments, the method may further include the addition of a unique molecular identifier (UMI) and/or randomer into the RNA adapter to facilitate further processes. In some embodiments, the UMI may be a PCR duplicate removal.

In some embodiments, the LNP-encapsulated RNA sample is treated with a phosphatase. In some embodiments, the LNP-encapsulated RNA sample is treated with FastAP. In some embodiments, the LNP-encapsulated RNA sample is pre-treated with FastAP. In some embodiments, the LNP-encapsulated RNA sample flexibility can be calculated by using sequencing or base calling errors. In some embodiments, the isolated RNA from the LNP can be calculated by using sequencing or base calling errors. In some embodiments, the LNP-encapsulated RNA flexibility can be calculated using mutation rates. In some embodiments, the mutation rates are calculated by running a solvent only as a control.

In some embodiments, the lipid the lipid nanoparticles may include one or more excipients selected from neutral lipids, charged lipids, steroids, and polymer conjugated lipids (e.g., pegylated lipid). In some embodiments, the lipid nanoparticles are not restricted to any particular morphology, and should be interpreted as to include any morphology generated when a cationic lipid and optionally one or more further lipids are combined, e.g., in an aqueous environment and/or in the presence of a nucleic acid compound. For example, a liposome, a lipid complex, a lipoplex, and the like are within the scope of a lipid nanoparticle. In some embodiments, the lipid nanoparticles may include any lipid capable of forming a particle to which the one or more nucleic acid molecules are attached, such as mRNA, or in which the one or more nucleic acid molecules are encapsulated.

In some embodiments, the lipid nanoparticles comprise one or more RNA (for example, mRNA) molecules that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%. In some embodiments, the lipid particle comprises RNA (e.g. mRNA) that is fully encapsulated within the lipid portion of the particles, from about 30%, 35%, 40%, 45%, 50%, 55%, 60%, 65%, 70%, 75%, 80%, 85%, 90%, 91%, 92%, 93%, 94%, 95%, 96%, 97%, 98%, 99% or ranges including and/or spanning the aforementioned values of the particles have the RNA encapsulated therein.

In some embodiments, the lipid nanoparticles include a lipid conjugate. “Lipid conjugate” means a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers, and mixtures thereof. PEG or POZ can be conjugated directly to the lipid or may be linked to the lipid via a linker moiety. Any linker moiety suitable for coupling the PEG or the POZ to a lipid can be used including, e.g., non-ester-containing linker moieties and ester-containing linker moieties. In some embodiments, non-ester-containing linker moieties, such as amides or carbamates, are used.

In some embodiments, the lipid nanoparticles include an amphipathic lipid. “Amphipathic lipid” means the material in which the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

In some embodiments, the lipid nanoparticles include a neutral lipid. “Neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

In some embodiments, the lipid nanoparticles include a non-cationic lipid. “Non-cationic lipid” means an amphipathic lipid or a neutral lipid or anionic lipid, and is described in more detail below.

In some embodiments, the lipid nanoparticles include an anionic lipid. “Anionic lipid” means a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, the lipid nanoparticles include a hydrophobic lipid. “Hydrophobic lipids” means compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacryloxy-3-aminopropane, and 1,2-dialkyl-3-aminopropane.

In some embodiments, the lipid nanoparticles include a cationic lipid and amino lipid. “Cationic lipid” and “amino lipid” are used interchangeably mean those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKₐ of the cationic lipid and is substantially neutral at a pH above the pKₐ. The cationic lipids of the disclosure may also be termed titratable cationic lipids. In some embodiments, the cationic lipids comprise: a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, (15Z, 18Z)—N,N-dimethyl-6-(9Z,12Z)-octadeca-9,12-dien-1-yl) tetracosa-15,18-dien-1-amine, (15Z, 18Z)—N,N-dimethyl-6-((9Z,12Z)-octadeca-9,12-dien-1-yl) tetracosa-4,15,18-trien-1-amine, (15Z,18Z)—N,N-dimethyl-6-((9Z,12Z)-octadeca-9,12-dien-1-yl) tetracosa-5,15,18-trien-1-amine, DSDMA, DODMA, DLinDMA, DLenDMA, γ-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA) (also known as 1-Bl 1). In some embodiments, the cationic lipid includes a commercially available cationic lipid. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the lipid nanoparticles include monocationic lipids. Monocationic lipids include, but are not limited to, 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradecoxy)propyl-(2-hydroxyethyl)-dimethylazanium bromide (DMRIE), didodecyl(dimethyl)azanium bromide (DDAB), 1,2-dioleyloxypropyl-3-dimethyl-hydroxyethyl ammonium bromide (DORIE) or 3β-[N—(N\N′-dimethylamino-ethane)carbamoyl]cholesterol (DC-Chol).

In some embodiments, the lipid nanoparticles include a lipid composition, comprising a nanoparticle or a bilayer of lipid molecules. In some embodiments, the lipid bilayer further includes a neutral lipid or a polymer. In some embodiments the lipid composition further encapsulates a nucleic acid. In some embodiments, the lipid composition further includes a nucleic acid and a neutral lipid or a polymer. In some embodiments, the lipid composition encapsulates the nucleic acid. In some embodiments, the nucleic acid is one or more RNA molecules. In some embodiments, the one or more RNA molecules includes a nucleotide sequence homologous to an mRNA in a target cell. In some embodiments, the nucleic acid is one or more therapeutic mRNA molecules encapsulated within the lipid particles.

In some embodiments, the one or more RNA molecules (e.g., mRNA molecules) are fully encapsulated within the lipid portion of the lipid particles such that the RNA in the lipid particles is resistant in aqueous solution to nuclease degradation. In other embodiments, the lipid particles described herein are substantially non-toxic to mammals such as humans. In some embodiments, the lipid particles have a mean diameter of from 30 nm to 150 nm, from 40 nm to 150 nm, from 50 nm to 150 nm, from 60 nm to 130 nm, from 70 nm to 110 nm, or from 70 to 90 nm. In some embodiments, the lipid particles have a lipid:RNA ratio (mass/mass ratio) of from 1:1 to 100:1, from 1:1 to 50:1, from 2:1 to 25:1, from 3:1 to 20:1, from 5:1 to 15:1, or from 5:1 to 10:1, or from 10:1 to 14:1, or from 9:1 to 20:1. In one embodiment, the lipid particles have a lipid:RNA ratio (mass/mass ratio) of 12:1. In another embodiment, the lipid particles have a lipid:RNA ratio (mass/mass ratio) of 13:1.

In some embodiments, the lipid particles comprise one or more RNA molecules (for example, mRNA molecules), a cationic lipid (for example, one or more cationic lipids or salts thereof described herein), a phospholipid, and a conjugated lipid that inhibits aggregation of the particles (for example, one or more PEG-lipid conjugates). The lipid particles can also include cholesterol. The lipid particles may comprise at least 1, 2, 3, 4, 5, 6, 7, 8, 9, 10, or more RNA that express one or more polypeptides.

In some embodiments, the nucleic acid-lipid particles, the one or more RNA molecules (e.g., mRNA) may be fully encapsulated within the lipid portion of the particle, thereby protecting the nucleic acid from nuclease degradation. In some embodiments, nucleic acid (e.g., mRNA) is fully encapsulated within the lipid portion of the lipid particles, thereby protecting the nucleic acid from nuclease degradation. In certain instances, the mRNA in the lipid particles is not substantially degraded after exposure of the lipid particles to a nuclease at 37° C. for at least 20, 30, 45, or 60 minutes. In certain other instances, the mRNA in the lipid particles is not substantially degraded after incubation of the particle in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the mRNA is complexed with the lipid portion of the particles. One of the benefits of the formulations of the present disclosure is that the nucleic acid-lipid particle compositions are substantially non-toxic to mammals such as humans. “Fully encapsulated” means that the nucleic acid (e.g., mRNA molecule) in the nucleic acid-lipid particle is not significantly degraded after exposure to serum or a nuclease assay that would significantly degrade free RNA. In some embodiments, when fully encapsulated less than 25% of the nucleic acid in the particle is degraded in a treatment that would normally degrade 100% of free nucleic acid. In some embodiments, when fully encapsulated less than 10% of the nucleic acid in the particles is degraded in a treatment that would normally degrade 100% of free nucleic acid. In some embodiments, when fully encapsulated less than 5% of the nucleic acid in the particles is degraded in a treatment that would normally degrade 100% of free nucleic acid. As used herein, “fully encapsulated” also means that the nucleic acid-lipid particles do not rapidly decompose into their component parts upon in vivo administration. In some embodiments, lipid nanoparticles include one or more lipids which stabilize the formation of particles during their formation. In some embodiments, suitable stabilizing lipids include neutral lipids and anionic lipids.

In some embodiments, the lipid nanoparticles have a mean diameter from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm. In some embodiments, the lipid nanoparticles include a lipid particle having a mean diameter from about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or ranges including and/or spanning the aforementioned values.

In some embodiments, the RNA molecule to be detected comprises a plurality of different RNA species, including without limitation, a plurality of different mRNA species, which may or may not be fragmented prior to generating ligation products. In some embodiments the RNA is fragmented chemically, enzymatically, mechanically, by heating, or combinations thereof. The term chemical fragmentation is used in a broad sense herein and includes without limitation, exposing the sample comprising the RNA to metal ions, for example, but not limited to, zinc (Zn²⁺), magnesium (Mg²⁺), and manganese (Mn²⁺) and heat. The term enzymatic fragmentation is used in a broad sense and includes combining the sample comprising the RNA with a peptide comprising nuclease activity, such as an endoribonuclease or an exoribonuclease, under conditions suitable for the peptide to cleave or digest at least some of the RNA molecules. Exemplary nucleases include without limitation, ribonucleases (RNases) such as RNase A, RNase T1, RNase T2, RNase U2, RNase PhyM, RNase III, RNase PH, ribonuclease V1, oligoribonuclease (e.g., EC 3.1.13.3), exoribonuclease I (e.g., EC 3.1.11.1), and exoribonuclease II (e.g., EC 3.1.13.1), however any peptide that catalyzes the hydrolysis of an RNA molecule into one or more smaller constituent components is within the contemplation of the current teachings. Fragmentation of RNA molecules by nucleic acids, for example, but not limited to, ribozymes, is also within the scope of the current teachings. The term mechanical fragmentation is used in a broad sense and includes any method by which nucleic acids are fragmented upon exposure to a mechanical force, including without limitation, sonication, collision or physical impact, and shear forces.

In some embodiments, the method further includes fragmenting mRNA. In some embodiments, the method further includes fractionating RNA that can be fragmented and analyzed using methods provided herein. In some embodiments, wherein fragmenting mRNA is performed by the group consisting of heating the RNA sample, treatment with RNase, addition of metal ions, or a combination thereof.

In some embodiments, the method further comprises isolating RNAs involved in translation of proteins within a cell (hereinafter “translation associated RNAs”). In some embodiments, the method further comprises isolating the translation associated RNAs. This step may be carried out by various methods such as a proximity ligation reaction. In some embodiments, the method further comprises amplifying enriched chimeric RNA molecules, or cDNA molecules thereof. In some embodiments, the method further comprises sequencing the PCR products. In some embodiments, the method further comprises identifying computationally chimeric RNA molecules.

In some embodiments, the target RNA sample may be taken from cells or tissue. Some embodiments further include lysing cells prior to isolating the complexes formed from the RNA and ribosomes. During the lysing process, cells may be incubated with lysis buffer and sonicated. In some embodiments, the lysing process further includes using RNase, such as RNase I, to partially fragment RNA molecules.

In some embodiments, beads can be added to an embodiment of the methods described herein. In some embodiments, the beads may be approximately 1 μm in size. In some embodiments, the beads may be silane beads. In some embodiments, the beads may be magnetic beads. In some embodiments, the beads may be silica magnetic beads. In some embodiments, the beads may be superparamagnetic particles with a bound protein. In some embodiments, the bound protein may be selective for biotin. In some embodiments, the bound protein is Streptavidin. In some embodiments, the beads are streptavidin magnetic beads. In some embodiments, the bound protein may be selective for antibodies. In some embodiments, the bound protein may be selective for anti-IgG. In some embodiments, the beads are dynabeads. In some embodiments, the beads are anti-rabbit dynabeads. In some embodiments, the bead is a BcMag magnetic bead. In some embodiments, the beads are monoavidin magnetic beads. In some embodiments, an on-bead probe can be added to an embodiment described herein. In some embodiments, the on-bead probe can target and enrich libraries in chimeric reads specific to one or more RNA of interest.

In some embodiments, a method may further include an enrichment step. In some embodiments, the enrichment step increases a proportion of unique mapped reads. In some embodiments, the enrichment step may produce reads out of all uniquely mapped reads of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or ranges including and/or spanning the aforementioned values. In some embodiments, the enrichment step may produce 5% to 30% chimeric reads out of all uniquely mapped reads.

In some embodiments, the sequences of RNA molecules are known, so probes can be designed to specifically bind to those RNA molecules. Such probes can specifically bind to non-chimeric RNA molecules, as well as RNA-target antibody oligo RNA chimeric molecules for enrichment. In some embodiments, the probes may be 100% complementary to the RNA molecules and in some cases the probes can include additional sequences to better cover imprecisely processed RNAs. In some embodiments, the mixture of RNA molecules is reverse transcribed into cDNA molecules before adding probes. In some embodiments, the probes are anti-sense nucleic acid probes having a length between 10 bp and 5 kb. In some embodiments, the probes are anti-sense nucleic acid probes in a length of 10 bp, 20 bp, 30 bp, 40 bp, 50 bp, 60, bp, 70 bp, 80 bp, 90 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1000 bp, 1100 bp, 1200 bp, 1300 bp 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, or ranges including and/or spanning the aforementioned values. In some embodiments, the probes may be between 10 bp and 1 kb, 10 bp and 500 bp, 10 bp and 250 bp, 10 bp and 100 bp, or 10 bp and 50 bp in length.

In some embodiments, the methods described herein can omit a gel clean up step. In some embodiments, omitting the gel clean up step may create a simplified high throughput version of the method.

In some embodiments, the RNA may be ligated with a reverse transcription adapter to facilitate formation of cDNA.

In some embodiments, the RNA may be contacted by an oligodT primer to facilitate formation of cDNA.

### Kits

Also provided by this disclosure are kits for practicing the methods as described herein. For example, the kit may contain a structure probing agent, media for resuspending the LNP-encapsulated RNA sample, DMSO, ligase, RNA or DNA oligos. In some embodiments, the kit may include one or more buffers and reagents. In some embodiments, the kit may include ssDNA Adapter. The ssDNA Adapter may include i7primer, DMSO, and bead elution buffer. In some embodiments, the kit may include an RT Adapter. In some embodiments, the kit may include one or more RT primers. The RT Adapter may include dNTPs and an RT Primer. In some embodiments, the kit may include a bead elution buffer. The bead elution buffer may include Tween 20, Tris buffer, and EDTA. In some embodiments, the kit may include library elution buffer. The library elution buffer may include Tris buffer, EDTA and sodium chloride. In some embodiments, the kit may include qPCR primers. In some embodiments, the kit may include PNK buffer. The PNK buffer may include Tris buffer, magnesium chloride, and ATP. In some embodiments, the kit may include an RT buffer. In some embodiments, the RT buffer may include Tris buffer, KCl, Manganese chloride, and DTT. In some embodiments, the kit may include bead binding buffer. The bead binding buffer may include RLT buffer and Tween 20. In some embodiments, the kit may include an RNA ligation buffer. The RNA ligation buffer may include Tris buffer, magnesium chloride, DMSO, Tween 20, ATP, and PEG. In some embodiments, the kit may include ssDNA ligation buffer. The ssDNA ligation buffer may include Tris buffer, magnesium chloride, DMSO, DTT, Tween 20, ATP and PEG8000. In some embodiments, the kit may include a mRNA Elution buffer. The mRNA Elution buffer may include Tris buffer and EDTA. In some embodiments, the kit may include a 2× Hybridization buffer. The 2× Hybridization buffer may include Tris buffer, lithium chloride, Tween 20 and EDTA.

### EXAMPLES

Examples are provided herein below. However, the presently disclosed and claimed inventive concepts are to be understood to not be limited in their application to the specific experimentation, results and laboratory procedures. Rather, the Examples are simply provided as one of various embodiments and are meant to be exemplary, not exhaustive.

### Example 1

In this example, an embodiment of a method for obtaining RNA structure by computing nucleotide flexibility for an LNP-encapsulated RNA of interest is described.

First, RNA-containing LNPs were obtained. Frozen LNPs were thawed at room temperature or reconstituted in 1×PBS if lyophilized. 100 ng of LNP-RNA was mixed with RNase A to remove any un-encapsulated RNA. The LNPs were then contacted either by 2-methylnicotinic acid imidazolide (NAI) or by a control chemical (vehicle) and incubated at 37° C. for RNA modification. An equal volume of 1M DTT was added to quench the NAI reaction. RNA was then isolated and cleaned from the reaction by adding magnetic beads and ethanol. Next, the RNA was fragmented in PNK buffer by heat.

Third, the fragmented RNA 3′ end was repaired using T4 PNK, leaving 3′-OH, which was needed for ligation.

Fourth, washed the previous step to remove the background.

Fifth, an RT primer-containing adapter was ligated to the 3′ end of the RNA.

Sixth, the previous reaction was washed for removal of unligated adapters.

Seventh, the RNA molecules were reverse transcribed to convert into CDNA.

Eight, the cDNA was cleaned up to prepare for step 9.

Ninth, the second adapter ligation was performed with UMI.

Tenth, the libraries were PCR amplified and cleaned up for sequencing.

Eleventh, the libraries made of the PCR products are sequenced.

Twelfth, the data was analyzed. The structure probing methods computes mutation rates that differed significantly from the control sample for the LNP-encapsulated RNA, indicating successful probing of encapsulated RNA (FIG. 2).

Further, the example above may have included trimming N10 UMIs from the 5′ ends of R1 reads and saved the UMI sequences in the read names to be utilized in subsequent steps, trimmed 3′ sequencing adapters, mapped reads to a reference genome, removed PCR duplicates by utilizing UMI sequences from the read names and mapped positions, computed mutation rates, reactivity scores for each single nucleotide, and applied the reactivity scores to determine single stranded areas of RNA for obtaining RNA structure.

### Example 2

This example is a protocol for structure probing a LNP-encapsulated RNA sample.

### Buffer Compositions and Reagents

ssDNA Adapter: 50 μL 100 μM i7primer, 60 μL DMSO, 140 μL Bead Elution Buffer
RT Adapter: 100 μL 10 mM dNTPs, 10 μl 10 μM RT Primer
Bead Elution Buffer: 0.001% Tween 20, 10 mM Tris pH 7.5, 0.1 mM EDTA
Library Elution Buffer: 20 mM Tris pH 7.5, 0.2 mM EDTA, 5 mM NaCl
qPCR Primers: 1.25 mM Primer 1, 1.25 mM Primer 2
RT Primer: 6.7 mM each dNTP, 3.3 μM RT Primer
PNK Buffer: 97.2 mM Tris pH 7, 13.9 mM MgCl2, 1 mM ATP
SHAPE RT buffer: 500 mM Tris-HCl, pH 8.0, 750 mM KCl, water
Bead Binding Buffer: 1×RLT buffer, 0.01% Tween 20
RNA Ligation Buffer: 75 mM Tris pH 7.5, 16.7 mM MgCl₂, 5% DMSO, 0.00067% Tween 20, 1.67 mM ATP, 25.7% PEG8000
ssDNA Ligation Buffer: 76.9 mM Tris pH 7.5, 15.4 mM MgCl₂, 3% DMSO, 30.8 mM DTT, 0.06% Tween 20, 1.5 mM ATP, 27.7% PEG8000
SHAPE folding buffer: 333 mM HEPES pH 8.0, 20 mM MgCl2, 333 mM NaCl, water.
2-methylnicotinic acid imidazolide (NAI): 2M NAI (in DMSO).

TABLE 1
Sequence

| | | SEQ |
|---|---|---|
| | | ID |
| Oligo | Sequence | NO |
| RT Primer | 5′-CAG ACG TGT GCT CTT C-3′ | 1 |
| ssDNA Adapter | 5′/5Phos/NNN NNN NNN NAG | 2 |
| | ATC GGA AGA GCG TCG TGT/ | |
| | 3SpC3/-3′ | |
| RNA Adapter | 5′-/SPhos/rArGrA rUrCrG | 3 |
| | rGrArA rGrArG rCrArC rArCrG | |
| | rUrCrU rG/iSpC3//3Bio/-3′ | |
| Q7 qPCR Primer | 5′-AGA CGT GTG CTC TTC CGA | 4 |
| | TCT-3′ | |
| Q5 qPCR Primer | 5′-TAC ACG ACG CTC TTC CGA | 5 |
| | TCT-3′ | |

TABLE 2
Reagent

| Vendor | Part # | Reagent |
|---|---|---|
| NEB | M0331B | 5′ Deadenylase |
| NEB | M0544B | NEBNext ® Ultra ™ II Q5 ® Master Mix |
| NEB | M0314B | RNase Inhibitor, Murine |
| NEB | M0201B | T4PNK with 10X PNK buffer |
| NEB | M0437B-BM | T4 RNA Ligase 1 (ssRNA Ligase) |
| Thermo Fisher | 18064022 | SuperScriptTM II Reverse Transcriptase |
| Thermo Fisher | EF0652B001 | FastAP Thermosensitive AP Supplied with: 10X |
| | | Buffer for fastAP - 10 × 1.5 mL |
| Thermo Fisher | 37005D | Dynabeads ™ MyOne ™ SILANE |
| Corning | 21-040-CV | PBS |
| Thermo Fisher | EN0531 | Rnase A |
| Thermo Fisher | 75001.10.ML | ExoSAP-IT Express PCR Product Cleanup Reagent |
| Millipore Sigma | 03-310 | NAI In vivo SHAPE reagent |
| Thermo Fisher | 011563.36 | Manganese Chloride |

### Library Prep:

### Preparation

Thawed structure probing agent and agent vehicle (control).

### Procedure

### Structure Probing

LNPs were thawed at room temperature. 100 ng LNP-RNA was moved to a new tube, and molecular biology grade water was added up to 9 μL. 3 μL RNase A (0.7 mU/μL) was added to the LNP-RNA. Next, 6 μL SHAPE folding buffer was added to the LNP-RNA sample. 2 μL 2M NAI was added to the probing sample, while 2 μL DMSO was added to the control sample. The probing mixtures were incubated at 37° C. for 15 minutes. The reaction was quenched by adding an equal volume (20 μL) 1M DTT. RNA was cleaned and isolated from the probing reactions using Silane magnetic beads. Eluted in 11 μL molecular biology grade water.

### RNA Fragmentation and End Repair

First, 2 μL 10×PNK Buffer was added to the ~9 μL of the eluted sample. The sample was heated to 98° C. for 2:30 minutes. Next, 1 μL of RNase inhibitor and 3 μL of PNK enzyme were added to the sample and incubated at 37° C. for 20 minutes.

### RNA Cleanup: 100 Nucleotide Cutoff Protocol

The sample volume was ~14 μL. 10 μL silane beads were cleaned using 5× volume bead binding buffer. Silane beads were resuspended in 93 μL bead binding buffer, per sample. 90 μL of resuspended beads were added to each sample, along with 60 μL 100% ethanol. Samples were mixed by pipetting and incubated at room temperature for 10 minutes. Samples were magnetized, the supernatant discarded, and 200 μL 80% ethanol was added to each sample. Samples were incubated for 30 seconds, the supernatant was discarded. The wash step was repeated twice for a total of 3 washes. Finally, the supernatant was completely removed, and beads were allowed to dry at room temperature for ~5 minutes. Beads were eluted in 8 μL Molecular biology grade water.

### RNA 3′ End Adapter Ligation

First, 5 μL of eluted RNA sample was transferred to new 0.2 mL strip tubes. The sample was kept on ice. 2 μL of RNA Adapter was added to each sample, pipette mixed, and incubated at 65° C. for 2 minutes. The sample was placed on ice after incubation. Next, the RNA Ligation Master Mix was prepared according to Table 3, below.

TABLE 3
RNA Ligation Master Mix.

| Reagent | Volume (μL) |
|---|---|
| RNA Ligation Buffer | 12 |
| RNase Inhibitor | 0.3 |
| T4 Ligase | 1.2 |
| Total: | 13.5 |

Second, 13.5 μL of the RNA Ligation Master Mix was added to each sample, pipette mixed, and incubated at room temperature for 45 minutes while rotating.

### RNA Silane Bead Cleanup

First, 10 μL sample Silane Beads (per sample) were added to a new 1.5 mL LoBind tube. 5× volume Bead Binding Buffer (per sample) was added to the tube with beads. The sample was pipette mixed until homogenous. The sample was placed on a DynaMag-2 Magnet. When separation was complete and the supernatant was clear, the supernatant was carefully aspirated and discarded. Tubes were removed from the magnet and 63 μL Bead Binding Buffer (per sample) was added to the tube with beads, and pipette mixed until homogenous. 60 μL of Silane beads were transferred to each tube of ligated RNA sample. Sample was mixed until homogenous. 45 μL 100% Ethanol was added to each sample, sample was pipette mixed until homogenous. The sample was incubated at room temperature for 10 minutes, pipette mix after 5 minutes. The sample tubes were placed on magnet for ~30 seconds, until separation was complete. The supernatant was aspirated and discarded. 200 μL 80% ethanol was added to each sample, and sample was moved around on the magnet to carefully move beads within the sample without pipetting. The supernatant was carefully aspirated and discarded. Wash steps were repeated for a total of 3 washes. Next, the samples were capped and centrifuged on a mini-centrifuge for 10 seconds, to collect all beads and liquids. The tubes were placed on the magnet, and all liquids were aspirated. Beads were allowed to dry for ~5 minutes on the magnet at room temperature. Once dry, 11 μL Bead Elution Buffer was added to the beads, and the sample was pipette mixed until homogenous. Sample was incubated at room temperature, off the magnet, for 5 minutes. After incubation the sample was placed back on the magnet for 30 seconds, until the supernatant in transparent. While on magnet, the entire sample (~9 μL) was transferred from the beads to a new 0.2 mL strip tube. Sample was placed on ice, beads were discarded.

### Reverse Transcription of Sample Reagent Preparation

First, RNA sample, 9 μL was transferred into a new, labeled 0.2 mL strip tube. Second, added 1.5 μL of RT Primer into RNA. Third, mixed, and spun all samples in minicentrifuge for 5 seconds to draw all liquid to the bottom of the tube. Fourth, incubated at 65° C. for 2 minutes in thermal cycler with the lid preheated to 70° C. Fifth, after incubation, immediately transferred to ice for 1 minute. Sixth, the thermal cycler block was adjusted temperature to 54° C.-20 minutes (with lid set to 65° C.).

### Reverse Transcription of RNA

First, Reverse Transcription Master Mix was prepared according to Table 4 in a fresh 1.5 mL LoBind tube. Second, pipetted sample up and down 10 times to mix. Third, samples were stored on ice until use. Note: Included 3% excess volume to correct for pipetting losses.

TABLE 4
Reverse Transcription Master-Mix (per sample)

| Component | Volume (μL) |
|---|---|
| SHAPE RT Buffer | 2.0 |
| RNase Inhibitor | 0.4 |
| Superscript II | 0.6 |
| Molecular Biology Water | 5.76 |
| MnCl₂ | 0.24 |
| 100 mM DTT | 1 |
| Total: | 10 |

Fourth, 10 μL of the Reverse Transcription Master Mix was added to each sample leaving the samples on ice and pipetted to mix. Fifth, samples were spun in mini-centrifuge for 5 seconds to draw all liquid to the bottom of the tube. Sixth, samples were incubated at 45° C. for 3 hours in thermal cycler with the lid at 65° C. Seventh, after incubation, the samples were immediately placed on ice. Eighth, adjusted the thermal cycler block temperature to 37° C.-15 minutes (with the lid set to 45° C.).

cDNA End Repair of Samples

First, 2.5 μL of ExoSap-IT was added to each sample. Second, spun samples in mini-centrifuge for 5 seconds to draw all liquid to the bottom of the tube. Third, incubated in thermal cycler at 37° C. for 15 minutes with the lid at 45° C. Fourth, the strip-tube was removed and placed on ice. Fifth, adjusted the thermal cycler block to 70° C. —10 minutes (with lid set to 75° C.). Sixth, 1 μL of 0.5 M EDTA (pH 8) was added to each sample. Seventh, the samples were pipetted up and down gently 5 times to mix. Eighth, 3 μL of 1 M NaOH was added to each sample. Ninth, pipetted the samples up and down gently 5 times to mix. Tenth, the tubes were incubated at 70° C. for 10 minutes in thermal cycler with the lid at 75° C. Eleventh, strip-tubes were placed on ice for 10 seconds. Twelfth, 3 μL of 1 M HCl was added to each sample. Thirteenth, proceeded immediately to the next step.

cDNA Sample Bead Cleanup

Preparation Note: ssDNA Adapter and ssDNA Ligation Buffer were thawed at room temperature until completely melted, then stored ssDNA Adapter on ice and ssDNA Ligation Buffer at room temperature. Preparation Note: Prepare fresh 80% ethanol in Molecular Biology Grade water in a fresh 50 mL tube if it was not done previously. Stored at room temperature for up to 1 week. Keep the tube closed tightly.

First, silane beads were taken out of 4° C. and resuspended until homogeneous. Second, Silane beads were washed prior to addition to samples. Third, for each cDNA sample, 5 μL of Silane beads was transferred to a new 1.5 mL DNA LoBind tube (e.g. for 4 samples transfer 20 μL of Silane beads). Fourth, 5× volume of Bead Binding Buffer (for example, for 4 samples, added 100 μL buffer to 20 μL of silane beads) was added to the beads and pipetted up and down sample until sample was homogeneous. Fifth, the tube was placed on DynaMag-2 magnet. When separation was complete and supernatant is clear, carefully aspirate and discard supernatant without disturbing beads. Sixth, the tube was removed from magnet. Seventh, the silane beads were resuspended in 93 μL of Bead Binding Buffer per sample. Eighth, the sample was pipetted up and down until beads were fully resuspended. Ninth, 90 μL of washed silane beads were added to each cDNA sample. Tenth, the sample was pipetted up and down to mix until sample was homogeneous. Eleventh, 90 μL of 100% EtOH was added to each cDNA sample. Twelfth, sample was pipetted to mix until homogeneous. Thirteenth, sample was incubated at room temperature for 10 minutes, with pipette mixing every 5 minutes. Fourteenth, the samples were moved to fresh strip tube and a new labeled 0.2 mL strip tube was placed on a 96-well magnet and sample was transferred from old to new strip tube. Fifteenth, the sample was allowed to incubate for 1 minute or until separation was complete and liquid is transparent. Sixteenth, the supernatant was carefully discarded without disturbing the beads. Seventeenth, 150 μL of 80% EtOH was added. Eighteenth, the samples were moved to different positions on magnet to wash thoroughly. Nineteenth, an additional 150 μL of 80% EtOH was added. Twentieth, the sample was incubated on the magnet for 30 seconds until separation was completed and the supernatant is transparent. Twenty-first, supernatant was carefully aspirated and discarded while on magnet. Twenty-second, steps 17-21 were repeated once for a total of two washes. Twenty-third, capped samples were spun in a mini-centrifuge for 5 seconds to draw all the liquid to the bottom of the tube. Twenty-fourth, the tubes were placed back on the 96-well magnet. Twenty-fifth, the sample was incubated on the magnet for 10 seconds until separation was complete and the supernatant was transparent. Twenty-sixth, using fine tips, aspirated and discarded all the residual liquid without disturbing beads while on magnet. Twenty-seventh, the beads were allowed to air dry for 5 minutes or until the beads no longer appeared wet and shiny. Note: Do not over dry samples. Twenty-eighth, once completely dried, the tubes were carefully removed from magnet. Twenty-ninth, the beads were resuspended in 2.5 μL of ssDNA Adapter. Thirtieth, the sample was pipetted to mix until homogeneous. Thirty-first, the sample were incubated in thermal cycler at 70° C. for 2 minutes with the lid at 75° C. Thirty-second, following incubation, the samples were immediately placed on ice for 1 minute.

cDNA Ligation on Beads

First, cDNA Ligation master mix was prepared according to Table 5 in a fresh 1.5 mL LoBind tube. The mix was pipetted to combine (do not vortex). Used immediately. Note: Included 3% excess volume to correct for pipetting losses.

TABLE 5
cDNA Ligation Master Mix (per sample).

| Component | Volume (μL) |
|---|---|
| ssDNA Ligation Buffer | 6.5 |
| T4 Ligase | 1 |
| Deadenylase | 0.3 |
| Total: | 7.8 |

Second, 7.8 μL of cDNA Ligation master mix was slowly added to each sample from previous section (cDNA Bead Clean Up) and pipetted the mix until homogeneous. Third, the sample was incubated at room temperature overnight on a tube rotator.

Ligated cDNA Sample Cleanup

First, the ligated-cDNA samples from tube rotator were obtained. Second, to each cDNA sample, 5 μL of Bead Elution Buffer was added. Third, 45 μL of the Bead Binding Buffer was added and was pipetted to mix. Fourth, 45 μL of 100% EtOH was added to each sample and pipette mixed until homogeneous. Fifth, the samples were incubated at room temperature for 10 minutes, with pipette mixing every 5 minutes. Sixth, the strip-tubes were placed on the 96-well magnet and allowed to incubate for 1 minute or until the separation was complete and the liquid was transparent. Seventh, the supernatant was carefully aspirated and discarded without disturbing the beads. Eighth, 150 μL of 80% EtOH was added without disturbing the beads. Ninth, samples were moved to different positions on the magnet to wash thoroughly. Tenth, an additional 150 μL of 80% EtOH was carefully added. Eleventh, sample was incubated on magnet for 30 seconds or until separation was complete and the supernatant was transparent. Twelfth, the supernatant was carefully aspirated and discarded. Thirteenth, steps 7-11 were repeated for a total of two washes. Fourteenth, the capped samples were spun in mini-centrifuge for 3 seconds to draw all liquid to the bottom of the tube. Fifteenth, the tube was placed back on the 96-well magnet. Sixteenth, the sample was incubated on the magnet for 30 seconds or until the separation was complete and the supernatant was transparent. Seventeenth, while on magnet, all liquid was aspirated and discarded without disturbing the beads. Eighteenth, the beads were allowed to air dry for 5 minutes or until the beads no longer appeared wet and shiny. Nineteenth, once completely dry, the tubes were carefully removed from the magnet. Twentieth, 25 μL Bead Elution Buffer was added to each sample. Twenty-one, pipetted up and down to mix until sample was homogeneous. Twenty-two, the sample was incubated for 5 minutes at room temperature. Twenty-third, after incubation, samples were moved to 96-well magnet. Twenty-fourth, the sample were incubated on magnet for 30 seconds until separation was complete and supernatant was transparent. Twenty-fifth, the supernatant was transferred (containing eluted cDNA) to new 0.2 mL strip tubes.

Optional Stopping Point: If Stopping Here, Eluted cDNA Samples should be Stored at −80° C. Next Stopping Point: ~2 Hrs
cDNA Sample Quantification by qPCR

First, qPCR master mix was prepared for the appropriate number of reactions according to Table 6 in a fresh 1.5 mL LoBind tube. Note: Included 3% excess volume to correct for pipetting losses.

TABLE 6
qPCR quantification master mix (per sample)

| Component | Volume (μL) |
|---|---|
| NEB LUNA Universal | 5 |
| qPCR 2× Master Mix | |
| qPCR Primers | 4 |
| Total: | 9 |

Second, a 96- or 384-well qPCR reaction plate was obtained and labeled. Third, 1 μL of eluted cDNA samples was added to 9 μL of Molecular Biology Grade Water for a 1:10 dilution. Fourth, 9 μL of qPCR master mix was added into all assay wells on ice. Fifth, 1 μL of each diluted cDNA (or water for negative controls) was added into the designated well. Note: Stored remaining diluted cDNA samples on ice until PCR in the next section. Sixth, the plate was covered with a MicroAmp adhesive film and sealed with MicroAmp adhesive film applicator. Seventh, the plate was spun at 3,000×g for 1 minute. Eighth, the qPCR assay was run according to the user manual for the specific instrument with run parameters appropriate for SYBR. Note: For example, for the StepOnePlus qPCR system the appropriate program is: 95° C. —30 sec, (95° C. —10 sec, 65° C. —30 sec)×32 cycles; No melting curve. Ninth, qPCR Ct values were recorded for all samples. Tenth, the threshold was set to 0.5—this recommendation was for StepOnePlus System. Note: Typical acceptable Ct values range from 10 to 23. For robust estimation, Ct values for samples should be ≥10. If values were below 9, dilute the 1:10 diluted cDNA an additional 10-fold, and re-perform qPCR using the 1:100 diluted cDNA.

PCR Amplification of cDNA and Dual Index Addition

First, the index primers were thawed at room temperature until fully melted. The index primers were shook to mix and spun in mini-centrifuge for 3 seconds. The samples were stored on ice until use. Second, the PCR amplification was prepared reaction mix according to Table 7 in fresh 0.2 mL PCR strip-tubes. Tubes were kept on ice. Note: If samples were going to be multiplexed during high-throughput sequencing, ensure that all samples to be pooled together have a unique combination of indexing primers.

TABLE 7
PCR amplification reaction mix contents
(prepare individually for each sample)

| Component | Volume (μL) |
|---|---|
| Ligated cDNA | 16 |
| 501 Index Primer | 2 |
| 701 Index Primer | 2 |
| PCR mix | 20 |
| Total: | 40 |

Note in this embodiment, traditional Illumina ® index primers may be used.

Third, samples were pipette mixed to result in a combination. Fourth, combined samples were spun in mini-centrifuge for 3 seconds to draw all liquid to the bottom of the tube. Fifth, samples were kept on ice. Sixth, referred to Ct values recorded to calculate the appropriate number of cycles for each sample. Used formula to calculate N=Ct−6, where N was the number of cycles performed using the second (two-step) cycling conditions: N+6=Total cycles=Ct. Note: e.g. If Ct=13.1, then N=7 and Total number of PCR cycles equal 13 (6+7).

Seventh, the PCR for the specific number of cycles calculated for each sample was ran according to the PCR program shown in Table 8

TABLE 8
PCR Amplification program

| | | |
|---|---|---|
| Temperature | Time | Cycles |

| | | | |
|---|---|---|---|
| 98° C. | 30 | seconds | |
| 98° C. | 15 | seconds | 6 |
| 70° C. | 30 | seconds | |
| 72° C. | 40 | seconds | |

Extra N cycles (N = Ct value − 9)

| 98° C. | 15 | seconds | N* |
|---|---|---|---|
| 72° C. | 45 | seconds | |
| 72° C. | 1 | minute | |
| 4° C. | ∞ | | |
| Total number of PCR cycles | | | 6 + N |

*N should be ≥1 and <14.

Eighth, the samples were immediately put on ice to cool following PCR amplification.

### AMPure Library PCR Product Cleanup

Preparative Note: AMPure XP beads were allowed to equilibrate at room temperature for 5 minutes.

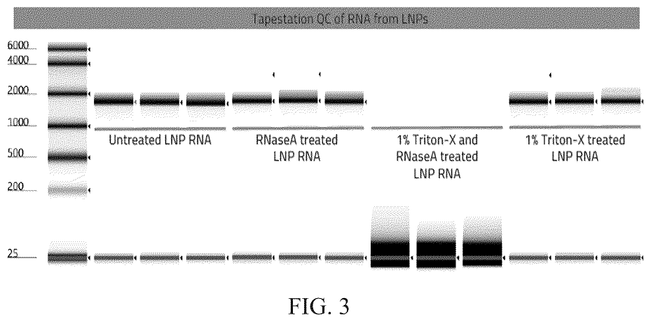

First, the AMPure XP beads were manually shook or vortexed to resuspend the sample until homogeneous. Second, 60 μL of AMPure XP beads were added into each 40 μL PCR reaction. Third, pipetted to mix until the sample was homogeneous. Fourth, the sample was incubated at room temperature for 10 minutes, with pipette mixing every 5 minutes. Fifth, the strip-tube was placed on the 96-well magnet and allowed to incubate for 1 minute or until separation was complete and the liquid was transparent. Sixth, the supernatant was carefully aspirated and discarded without disturbing the beads. Seventh, 150 μL of 80% EtOH was added without disturbing beads. Eighth, the samples were moved to different positions on magnet to wash thoroughly. Ninth, carefully added an additional 150 μL of 80% EtOH. Tenth, incubated on magnet for 30 seconds or until the separation was complete and the supernatant was transparent. Eleventh, carefully aspirated and discarded the supernatant. Twelfth, repeated steps 7-11 for a total of two washes. Thirteenth, the samples were spun in mini-centrifuge for 3 seconds to draw all liquid to the bottom of the tube. Fourteenth, tube was placed back on 96-well magnet. Fifteenth, incubated on the magnet for 30 seconds or until separation was complete and the supernatant was transparent. Sixteenth, while on the magnet, aspirated and discarded all the residual liquid without disturbing the beads. Seventeenth, the beads were allowed to air dry for 5 minutes or until the beads no longer appeared wet and shiny. Eighteenth, once completely dry, carefully removed tubes from the magnet. Nineteenth, 20 μL Molecular Biology Grade Water was added to each sample. Twentieth, pipetted the mix until sample was homogeneous. Twenty-first, the samples were incubated for 5 minutes at room temperature. Twenty-second, the samples were transferred the 20 μL of eluted sample to new strip-tube. Twenty-third, analyzed library length and concentration via Agilent Tapestation. Twenty-fourth, if adapter dimer was present, perform an agarose gel extraction for a DNA 200-400 nts in length and re-tapestation. FIGS. 2 and 3 illustrate results of the protocol described above.

What is claimed is:

1. A method of obtaining RNA secondary structure by assessing nucleotide flexibility of an RNA molecule within a lipid nanoparticle (LNP), the method comprising:
a) contacting an LNP-encapsulated RNA sample with one or more structure probing agents;
b) isolating at least one RNA or cDNA from the LNP-encapsulated RNA sample;
c) sequencing an isolated RNA or cDNA generated from the at least one RNA or cDNA; and
d) obtaining RNA secondary structure by assessing nucleotide flexibility of the LNP-encapsulated RNA sample.

2. The method of claim 1, wherein the LNP-encapsulated RNA sample is from mammal cells, bacterial cells, or viral particles.

3. The method of claim 1 or 2, wherein the LNP-encapsulated RNA sample is polyA-selected, ribosomal RNA depleted, in vitro transcribed, or of synthetic origin.

4. The method any one of claims 1-3, wherein the LNP-encapsulated RNA sample is linear or circular.

5. The method of any one of claims 1-4, wherein the LNP-encapsulated RNA sample comprises mRNA molecules.

6. The method any one of claims 1-5, wherein the one or more structure probing agents is capable of modifying RNAs 2′OH or one or more of RNAs nitrogenous bases.

7. The method of claim 6, wherein the one or more structure probing agents is NAI, NAI-N3, DMS, 1M7, 1M6, NMIA, 5NIA, 2A3, or combinations thereof.

8. The method of any one of claims 1-7, wherein the cDNA are formed by reverse transcribing RNA molecules into cDNA molecules before an enriching step.

9. The method of claim 8, wherein reverse transcribing RNA molecules comprises a reserve transcription buffer comprising manganese.

10. The method of claim 8, wherein reverse transcribing RNA molecules comprises a reverse transcriptase selected from the group consisting of SSII, SSIII, SSIV, TGIRT-III, MarathonRT, Maxima H, or combinations thereof.

11. The method of any one of claims 1-10, wherein the LNP-encapsulated RNA sample is treated with a phosphatase.

12. The method of claim 3, wherein the LNP-encapsulated RNA sample comprises one or more modified nucleic acids.

13. The method of claim 12, wherein the one or more modified nucleic acids is of synthetic origin selected from the group consisting of LNA (locked nucleic acid), pseudouridine, 1-methyl-pseudouridine, 6-methyl-adensoine, 2′ methyl, 2′-O-(2-methoxyethyl).

14. The method of any one of claims 1-13, wherein the LNP comprises at least one cationic lipid, non-cationic lipid, anionic lipid, stabilizing lipid, PEG, cholesterol, phospholipid, or combinations thereof.

15. The method of any one of claims 1-14, wherein RNA flexibility can be calculated by using sequencing or base calling errors.

16. The method of claim 15, wherein the RNA flexibility can be calculated using mutation rates.

17. The method of claim 16, where in the mutation rates are calculated by running a solvent only control.

18. The method of any one of claims 1-17, wherein the isolated RNA is generated from the LNP-encapsulated RNA sample by silane beads.

19. The method of any one of claims 1-18, wherein the one or more probing agents are quenched prior to RNA isolation.

20. The method of any one of claims 1-19, wherein the LNP-encapsulated RNA sample is treated with RNase.

21. The method of claim 20, wherein a separate RNA is spiked into the LNP-encapsulated RNA sample before treating with RNase to monitor digestion of RNAs not LNP-encapsulated.

22. A kit comprising:
one or more structure probing agents; and
a manual providing instructions for obtaining RNA secondary structure of an LNP-encapsulated RNA sample.

23. The kit of claim 22, further comprising one or more control RNAs.

24. The kit of claim 22 or 23, further comprising one or more buffers.

25. The kit of claim 24, wherein the one or more buffers is selected from the group consisting of bead elution buffer, FastAP, library elution buffer, PNK buffer, RT buffer, proteinase K buffer, bead binding buffer, RNA ligation buffer, and ssDNA ligation buffer.

26. The kit of any one of claims 22-25, further comprising one or more primers.

27. The kit of claim 26, wherein the one or more primers is selected from the group consisting of qPCR primer and RT primer.
